(19) United States
(12) Patent Application Publication
(10) Pub. No.: US 2005/0019914 A1
(43) Pub. Date: Jan. 27, 2005

(54) Perfusion process for producing erythropoietin

(75) Inventors: Andreas Staerk, Eppstein (DE); Klaus Scharfenberg, Emden (DE); Norbert Schulze, Hattersheim (DE); Kathrin Baumeister, Frankfurt (DE); Wilhelm Beltz, Biedenkopf (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 10/896,660

(22) Filed: Jul. 22, 2004

Related U.S. Application Data
(60) Provisional application No. 60/527,951, filed on Dec. 8, 2003.

(30) Foreign Application Priority Data
Jul. 24, 2003 (DE) 10333675.3-41

(51) Int. Cl. C12N 5/00; C12N 5/02

(52) U.S. Cl. 435/374

(57) ABSTRACT

The invention relates to a process for producing erythropoietin (EPO) in which eukaryotic cells, which are suitable for expressing EPO, are adapted to SMIF7 medium in a suitable bioreactor, the resulting cells are transferred to a larger bioreactor and further expanded with SMIF7 medium and, while constantly bleeding and constantly perfusing, the expressed EPO is isolated from the larger bioreactor and purified.

The invention relates to a process for producing erythropoietin (EPO) in which eukaryotic cells, which are suitable for expressing EPO, are adapted to SMIF7 medium in a suitable bioreactor, the resulting cells are transferred to a larger bioreactor and further expanded with SMIF7 medium, and a cell concentration steady state is achieved by a strategy of continuously perfusing and bleeding off cells.

Human erythropoietin (EPO) is composed of 165 amino acids and, because of the heterogeneity of its glycan structures, has an apparent mass of 34-39 kDa. The protein possesses three N-glycosylation sites at Asn24, Asn38 and Asn83 and one O-glycosylation site at Ser126 which are sialylated to a high degree.

The importance of the sialylation for the in vivo activity of EPO has been known for a long time and verified by many studies. As early as 1960, Lowy et al. (1960, Nature 185: 102-103) demonstrated that when EPO is desialylated, it loses its in vivo activity completely. In 1989 (Blood 73: 90-99) Spivak and Hogans found a highly sialylated EPO to have a half-life of 53 minutes. By comparison, the desialylated form only had a half-life of 3 minutes.

This means that, for producing EPO on an industrial scale, or for establishing a process for producing EPO, the demand is to achieve, by means of selecting suitable parameters, a degree of sialylation of the EPO which is as high as possible. The present process relates to producing EPO on the basis of a perfusion/bleeding strategy using crossflow filtration for cell retention.

Perfusion processes for expressing recombinant proteins on an industrial scale have been established for a long time and are described in a large number of publications.

The benefit of these production processes generally resides in very high space/time yields as compared with other strategies for conducting the process (batch and fed batch). For example, perfusion processes prevent toxic metabolites from becoming concentrated and substrate from becoming limited, thereby making it possible to achieve higher cell densities and higher vitalities, thereby ultimately ensuring longer production times and i.e. space/time yields.

Using such a production strategy for producing a glycoprotein, e.g. EPO, is particularly advantageous since the product is removed continuously from the process, thereby making it possible to minimize degradation brought about by glycosidases and proteases, etc. For the abovementioned reasons, it follows that such a production process should ensure the high degree of sialylation which EPO requires.

Depending on the cell line employed, and the specific requirements, crossflow filters (Prostak), spin filters, ultrasonic separators, centrifuges and inclined settlers are used as cell retention systems for achieving these strategies (Woodside et al. 1998, Mammalina cell retention devices for stirred perfusion bioreactors, Cytotechnology 28: 163-175; Castilho, L. R., Medronho, R. A. 2002, Cell retention devices for suspended-cell perfusion cultures, Adv Biochem Eng Biotechnol. 74: 129-169). The Prostak module which is used for retaining cells in the present production process, and which is based on crossflow filtration (tangential flow filtration), is also well documented in the literature. For example, the crossflow filtration which is used here has been employed to culture a variety of hybridoma cells and cancer cells (e.g. Kawahara et al. 1994, Cytotechnology 14: 61; de la Broise et al. 1992, Biotechnol Bioeng 40: 25). Although Prostak modules can be obtained commercially, there are few reports in the literature of their use on a pilot scale.

A perfusion/bleeding strategy which uses crossflow filtration up into the 100 l scale, which is adapted to the GA-EPO HT1080 cell (preparation described in detail in “Trial Exhibit No. 20, Amgen Inc. V. Hoechst Marion Roussel, Inc. and Transkaryotic Therapies, Inc; US District Court of Massachusetts C.A. 97-10814WGY”) and which ensures a robust culturing process of up to 32 production days, is described below. In this strategy, cell retention is achieved using a Prostak module which possesses three 10 stacks arranged in series (Millipore, Molsheim, France). When using this set-up, the maximum daily volumetric capacity of 100 l perfusion unit is 2.5 reactor volumes, corresponding to 250 L d⁻¹.

As was found during development, it is very difficult to establish a continuous perfusion process which fully exploits these system parameters since, when problems, such as the accumulation of toxic metabolites, arise, there is no scope for action (e.g. by raising the perfusion rate). Furthermore, the danger exists that the filters of the Prostak modules will block more rapidly due to higher cell densities, with this ultimately leading to more rapid termination of the process.

It has now been found, surprisingly, that high yields of EPO are obtained continuously using a perfusion/bleeding strategy in which the set-up only has a relatively low work-load and preference is given to using special culture media for culturing the cells. The process which is presented here describes a perfusion/bleeding strategy in which a cell density of approximately 1-3 E⁶ ml⁻¹ is maintained constantly in the production phase. Under the chosen conditions, this procedure involves an approximately 50% work-load, corresponding to about 125 L d⁻¹ of perfusate.

One part of the subject-matter of the invention is a process for producing erythropoietin (EPO) which comprises
(a) adapting eukaryotic cells, which are suitable for expressing EPO, to SMIF7 medium in a suitable bioreactor and expanding them to a cell density which is such that a cell density of from 2×10⁵ ml⁻¹ to 5×10⁵ ml⁻¹ is obtained in the subsequent step (b),
(b) transferring the cells obtained in step (a) to a larger bioreactor and culturing them using SMIF7 medium,
(c) expanding the cells cultured in the larger bioreactor to a cell density of from 1×10⁶ ml⁻¹ to 1×10⁷ ml⁻¹,
(d) using a perfusion/bleeding strategy to maintain a cell density of from 1×10⁶ ml⁻¹ to 1×10⁷ ml⁻¹ constant (steady state) and continuously removing the expressed EPO from the larger bioreactor and isolating and purifying it,
   with the eukaryotic cells, which are initially present in frozen form, preferably being revitalized using the medium DMEM/F12 1:1 and preferably being supplemented, per liter, like the SMIF7 medium as well, with from 1.5 to 2.5 g of NaHCO₃, particularly preferably from 2.0 to 2.3 g, very particularly preferably 2.16 g of NaHCO₃; from 0.2 to 5 g, particularly preferably from 0.5 to 2 g, very particularly preferably 1 g of BSA; from 0.2 to 5 mg, particularly preferably from 0.5 to 2 mg, very particularly preferably 1 mg of human transferrin; from 1 to 30 mg, particularly preferably from 5 to 15 mg, very particularly preferably 10 mg of human insulin; from 1 to 3 mg, particularly preferably 2 mg of hydrocortisone; from 0.01 to 0.1 mg, particularly preferably from 0.025 to 0.045 mg, very particularly preferably 0.039 mg of dexamethasone; from 0.08 to 3 mg, particularly preferably from 1.5 to 3 mg, very particularly preferably 2 mg of putrescine, from 40 to 100 mg, particularly preferably from 50 to 80 mg, very particularly preferably 60 mg of ethanolamine; from 200 to 500 mg, particularly preferably from 250 to 400 mg, very particularly preferably 292 mg of glutamine and from 50 to 100 mg, particularly preferably from 70 to 90 mg, very particularly preferably 80 mg of serine.

Another part of the subject-matter of the invention is a process as described above, with the volume of the bioreactor in step (a) being from 5 to 50 liters and/or the volume of the larger bioreactor in step (b) being from 50 to 200 liters.

Another part of the subject-matter of the invention is a process as described above with the eukaryotic cells in step (a) and/or step (c) being expanded to a cell density of from 1×10⁶ to 1×10⁷ ml⁻¹, preferably from 1×10⁶ to 3×10⁶ ml⁻¹, and the eukaryotic cells preferably being GA-EPO HT 1080 cells.

The subject-matter of the invention is explained in more detail below with the aid of an example without being restricted to this example.

### EXAMPLE

### Culturing an EPO-Producing Cell Line

The GA-EPO HT 1080 cells, which have been revitalized from a cryocell bank, are expanded, in DMEM/F12 1:1 (Invitrogen) which has been supplemented, per liter, with 2.16 g of NaHCO₃ (Merck), 1 g of BSA (Sigma), 1 mg of human transferrin (Chiron), 10 mg of human recombinant insulin (Aventis), 2 mg of hydrocortisone (Sigma), 0.039 mg of dexamethasone (Sigma), 2 mg of putrescine (Sigma), 60 mg of ethanolamine (Sigma), 1 g of Pluronic F68 (Sigma), 292 mg of glutamine (Fluka) and 80 mg of serine (Fluka), in T flasks and spinners and inoculated into a 20 l bioreactor. In this matter, the cells are readapted to SMIF7 medium (Invitrogen) which has been supplemented with the same chemicals and quantities, in analogy with DMEM F12 1:1. After an adaptation period of approx. 14 days, the cells are transferred to the 100 l bioreactor at a cell density of 3×E⁵ ml⁻¹. The minimal production parameters (cell density greater than equal to 1 E⁶ ml⁻¹ are achieved after approx. 9 days (Tab. 1, beginning of the production phase).

TABLE 1
Culturing GA-EPO cells using a perfusion/bleeding strategy.

| Culturing | Live cell count/ | Vitality/ | Bleeding/ | Perfusion/ | c_{EPO /} | cum · | Z |
|---|---|---|---|---|---|---|---|
| time/d | ×10⁵ ml⁻¹ | % | vvd | vvd | μg ml⁻¹ | EPO/g | number. |
| 0.1 | 3 | 77 | 0.00 | 0.0 | 29.4 | — | 293 |
| 1.9 | 4 | 75 | 0.00 | 0.0 | 17.4 | — | — |
| 4.9 | 4 | 79 | 0.00 | 0.0 | 15.8 | — | 312 |
| 5.9 | 5 | 76 | 0.00 | 0.0 | 18.3 | — | — |
| 6.9 | 5 | 75 | 0.00 | 0.3 | 16.3 | — | 288 |
| 7.9 | 6 | 79 | 0.00 | 0.3 | — | — | — |
| 8.9 | 10 | 80 | 0.00 | 0.7 | 14.3 | — | — |
| 11.9 | 12 | 81 | 0.00 | 1.0 | 12.1 | 3.40 | 305 |
| 12.9 | 14 | 76 | 0.00 | 1.3 | 8.9 | 5.50 | — |
| 13.9 | 17 | 85 | 0.14 | 1.5 | 8.9 | 7.78 | 334 |
| 14.9 | 16 | 83 | 0.14 | 1.5 | — | — | — |
| 15.9 | 18 | 81 | 0.15 | 1.6 | 8.3 | 9.84 | — |
| 18.9 | 8 | 82 | 0.15 | 1.6 | 13.1 | 15.70 | 306 |
| 19.9 | 13 | 86 | 0.00 | 1.3 | 6.3 | 17.20 | — |
| 20.9 | 23 | 85 | 0.38 | 1.6 | 10 | 19.87 | — |
| 21.8 | 22 | 79 | 0.38 | 1.6 | 6.4 | 20.75 | 334 |
| 22.9 | 21 | 85 | 0.15 | 1.3 | 12.1 | — | 312 |
| 24.2 | 26 | 84 | 0.15 | 1.3 | 14 | 22.50 | — |
| 25.9 | 19 | 84 | 0.24 | 1.3 | 12.5 | 24.64 | — |
| 26.9 | 21 | 82 | 0.15 | 1.3 | — | — | — |
| 27.9 | 22 | 81 | 0.20 | 1.3 | 13.5 | 27.76 | 324 |
| 28.9 | 17 | 84 | 0.20 | 1.3 | 12.5 | 30.76 | 278 |
| 29.9 | 20 | 80 | 0.16 | 1.3 | 12.3 | — | — |
| 31.3 | 23 | 84 | 0.20 | 1.3 | 11.3 | 33.42 | — |
| 32.9 | 19 | 81 | 0.25 | 1.3 | 7.72 | 35.81 | — |
| 33.9 | 16 | 79 | 0.14 | 1.3 | — | — | — |
| 34.9 | 24 | 83 | 0.25 | 1.3 | 14.2 | 38.16 | 327 |
| 36.0 | 24 | 84 | 0.25 | 1.3 | — | — | — |
| 36.9 | 27 | 86 | 0.24 | 1.3 | 17.7 | 42.21 | — |
| 38.3 | 26 | 86 | 0.27 | 1.3 | n.d. | — | — |
| 39.9 | 26 | 87 | 0.27 | 1.3 | 15.5 | 45.81 | 337 |
| 40.9 | 26 | 87 | 0.27 | 1.3 | 15.2 | 46.64 | — |

The stable production phase lasts 32 days. During this period, 4530 l of culture supernatant are produced, with this volume containing 46.64 g of EPO (C_{EPO} between 6.3 and 17.7 mg ml⁻¹). The quality of the EPO is assessed by analyzing the Z number. To do this, HPAE chromatography (high-pH anion-exchange chromatography) is used to separate the N-glycans on the basis of their charge (nonsialo, monosialo, etc.). In order to calculate the Z number, the respective peak areas are multiplied by their corresponding charge and the individual results are added up. The Z number thereby provides important information with regard to the sialylation status and the antennarity of N-glycans. Highly purified therapeutic agents comprising rhu EPO produced by the companies Roche Mannheim, Amgen, Organon Teknika and Merckle possess Z numbers of 361, 367, 286 and 323, respectively (Hermentin et al. (1996). The hypothetical N-glycan charge: a number that characterizes protein glycosylation. Glycobiology 6: 217-230).

Over its entire course, the perfusion process which is presented here yields Z numbers for the crude, unpurified EPO of between 278 and 337 (mean value 315.5). This is a high value for an unpurified EPO and therefore demonstrates the feasibility of the perfusion/bleeding strategy which has been presented.

1. A process for producing erythropoietin (EPO) which comprises
a. adapting eukaryotic cells, which are capable of expressing EPO, to SMIF7 medium in a bioreactor and expanding them to a cell density of from 5×10⁵ to 5×10⁶ ml⁻¹,
b. transferring the cells obtained in step (a) to a larger bioreactor and diluting them with SMIF7 medium to a cell density of from 1×10⁵ ml⁻¹ to 1×10⁶ ml⁻¹,
c. expanding the cells cultured in the larger bioreactor to a cell density of from 5×10⁵ to 5×10⁶ ml⁻¹,
d. bleeding culture supernatant from the cells from step (c) while perfusing with SMIF7 medium, and
e. isolating and purifying the expressed EPO from the bled culture supernatant.

2. The process as claimed in claim 1, wherein the step of adapting eukaryotic cells comprises revitalizing eukaryotic cells from a frozen form in DMEM/F12 1:1 medium before transferring the revitalized cells to the SMIF7 medium for expansion to a cell density of from 5×10⁵ to 5×10⁶ ml⁻¹.

3. The process as claimed in claim 2, wherein the medium for the revitalization and the SMIF7 medium are supplemented, per liter, with from 1.5 to 2.5 g of NaHCO₃; from 0.2 to 5 g of BSA; from 0.2 to 5 mg of human transferrin; from 1 to 30 mg of human insulin; from 1 to 3 mg of hydrocortisone; from 0.01 to 0.1 mg of dexamethasone; from 0.08 to 3 mg of putrescine, from 40 to 100 mg of ethanolamine; from 200 to 500 mg of glutamine and from 50 to 100 mg of serine.

4. The process as claimed in claim 2, wherein the medium for the revitalization and the SMIF7 medium are supplemented, per liter, with from 2.0 to 2.3 g of NaHCO₃; from 0.5 to 2 g of BSA; from 0.5 to 2 mg of human transferrin; from 5 to 15 mg of human insulin; from 1 to 3 mg of hydrocortisone; from 0.025 to 0.045 mg of dexamethasone; from 1.5 to 3 mg of putrescine, from 50 to 80 mg of ethanolamine; from 250 to 400 mg of glutamine and from 70 to 90 mg of serine.

5. The process as claimed in claim 2, wherein the medium for the revitalization and the SMIF7 medium are supplemented, per liter, with 2.16 g of NaHCO₃; 1 g of BSA; 1 mg of human transferrin; 10 mg of human insulin; 2 mg of hydrocortisone; 0.039 mg of dexamethasone; 2 mg of putrescine, 60 mg of ethanolamine; 292 mg of glutamine and 80 mg of serine.

6. The process as claimed in one of the preceding claims, wherein the volume of the bioreactor in step (a) is from 5 to 50 liters.

7. The process as claimed in one of the preceding claims, wherein the volume of the larger bioreactor in step (b) is from 70 to 200 liters.

8. The process as claimed in one of the preceding claims, wherein the eukaryotic cells in step (a) and/or step (c) are expanded to a cell density of from 8×10⁵ to 2×10⁶ ml⁻¹, preferably of 1×10⁶ ml⁻¹.

9. The process as claimed in one of the preceding claims, wherein the eukaryotic cells are GA-EPO HT 1080 cells.
